(19) United States
(12) Patent Application Publication
(10) Pub. No.: US 2013/0017556 A1
(43) Pub. Date: Jan. 17, 2013

(54) ASSAYS FOR HDL BIOMOLECULAR INTERACTIONS

(71) Applicant: Kirkwood A. Pritchard Jr., JR., Elm Grove, WI (US)

(72) Inventor: Kirkwood A. Pritchard Jr., JR., Elm Grove, WI (US)

(21) Appl. No.: 13/447,532

(22) Filed: Apr. 16, 2012

Related U.S. Application Data
(60) Provisional application No. 61/478,279, filed on Apr. 22, 2011.

(51) Int. Cl. G01N 33/573; G01N 21/21; G01N 21/45; G01N 33/566; G01N 21/55

(52) U.S. Cl. 435/7.4; 436/501

(57) ABSTRACT

The invention relates to methods, compositions and kits for detecting molecular interactions. In one embodiment, the invention relates to methods for quantifying HDL interactions with biomolecules. In still another embodiment, the invention relates to methods for determining HDL function.

### CROSS-REFERENCE TO RELATED APPLICATIONS

This application claims priority under 35 U.S.C. §119(e) to U.S. Provisional Patent Application No. 61/478,279 filed Apr. 22, 2011, the entirety of which is incorporated by reference herein.

### STATEMENT REGARDING FEDERALLY SPONSORED RESEARCH OR DEVELOPMENT

This invention was made with United States government support awarded by the National Institutes of Health, grant Nos. HL081139 and HL 102836. The United States government has certain rights in this invention.

### FIELD

Embodiments of the invention relate to the fields of cell biology, molecular biology and therapeutics. More specifically, embodiments of the invention are related to methods, compositions and kits for assaying interactions between biomolecules. In another embodiment, the invention relates to methods, compositions, and kits for determining a binding rate between molecules.

### BACKGROUND

Arteriosclerosis, which literally means “hardening of the arteries,” actually refers to a group of disorders that involves a thickening and loss of arterial elasticity. Although they frequently occur together, each of the principal disorders (Monckeberg's medial calcific sclerosis, arteriolosclerosis, and atherosclerosis) are distinguishable by the afflicted artery's morphological appearance. Monckeberg's medial calcific sclerosis is characterized by ringlike calcifications in small to medium sized arteries. Arteriolosclerosis is characterized by a thickening of artery and arteriole walls, resulting in lumen narrowing.

The predominant and most serious form of arteriosclerosis is atherosclerosis. In Western countries, atherosclerosis is responsible for 20% to 25% of myocardial infarction deaths yearly, and is a contributing factor in about 50% of deaths from all other causes. Atherosclerosis is also the major cause of a large number of morbidities, including chronic ischemic heart disease, gangrene, mesenteric occlusion, and ischemic encephalopathy.

Atherosclerosis is believed to begin in childhood as a progressive disease that first strikes the large- and medium-sized arteries. These include the coronary, the carotid, the aorta, and the larger arteries of the lower extremities. Later in life, lesions called “atheromas” or “fibrofatty plaques” form on the arterial inner walls. These plaques have a central necrotic core of lipid deposits composed primarily of cholesterol, calcium, cellular debris, and other materials. As the disease progresses, plaques coalesce, forming large masses, or what have been called “complicated plaques,” in which there may be associated arterial calcification. Fatty streaks appear on the vessel walls, causing them to ulcerate and rupture. Debris (such as cholesterol emboli) is then released into the bloodstream. Hemorrhaging may occur from rupture of the overlying capillary endothelium. Eventually, clotting may occur within the vessel, causing tissue infarction.

The major risk factors associated with heart disease include hypercholesterolemia, hypertension, hyperglycemia and smoking. Hypercholesterolemia (high serum cholesterol) is present most of the time, and serum cholesterol concentrations correlate with mortality.

For years it has been recognized that HDL cholesterol was inversely related to risk of atherosclerosis. However, re-analysis of data from the Framingham Heart studies revealed that nearly 40% of clinically significant events actually occurred in subjects who had normal levels of HDL cholesterol (Gordon T, et al., Am J Med. 1977; 62:707-714). Furthermore, analysis of the Air Force/Texas Coronary Prevention Study showed that event rates in individuals with normal HDL levels, who also received placebo, were nearly two-thirds the event rates in individuals with the lowest HDL levels (<34 mg/dL), the group most at risk (Ansell B J, et al., Circulation, 2003; 108:2751-2756; Downs J R, et al., JAMA. 1998; 279:1615-1622). These clinical studies provided strong support for the idea that HDL protects cardiovascular health by mechanisms that do not appear to correlate with the cholesterol content of HDL (HDL-C), the main biomarker currently used to monitor risk in patients.

Therefore, it would be useful to identify methods, compositions and kits that can assay HDL interactions with other biomolecules.

### BRIEF SUMMARY

The invention relates methods, compositions, and kits to assay HDL interactions with biomolecules. In one embodiment, the invention relates to an analytical assay for quantifying HDL interactions with other biomolecules. In yet another embodiment, the invention relates to methods and compositions for determining one or more binding rates between HDL and a biomolecule. The biomolecules can be associated with inflammation, cholesterol transport, reverse cholesterol transport, cholesterol metabolism, oxidation, an anti-oxidant enzyme, and a pro-oxidant enzyme.

In another embodiment, invention relates to methods for determining HDL function. In yet another embodiment, the invention relates to methods for correlating HDL binding with HDL function. In still another embodiment, the methods, compositions and kits relate to determining a risk for atherosclerosis.

In one embodiment, the invention relates to a method comprising: providing immobilized HDL on a surface; bringing said surface into contact with a sample under conditions that are permissive of binding of a biomolecule in said sample to an HDL molecule; and detecting whether biomolecule-HDL binding occurs upon said contact.

In another embodiment, the invention relates to a method comprising: contacting a surface with immobilized HDL with a sample containing a biomolecule; detecting HDL-biomolecule binding on said surface; and correlating biomolecule-HDL binding with HDL function.

In another embodiment, the invention relates to a method for measuring binding of a biomolecule to high density lipoprotein (HDL) comprising: (a) providing a biosensor with two reflecting surfaces, wherein interference between a reflected beam from a first reflecting surface, which comprises immobilized HDL, and a reflected beam from a second reflecting surface, which lacks immobilized HDL, varies upon binding of a biomolecule to HDL; (b) exposing said biosensor to a biomolecule; and (c) determining whether a change occurs in the interference between the reflected beams, whereby such a change is indicative of binding kinetics between the biomolecule and HDL.

In another embodiment, the method further comprises determining a binding rate of a biomolecule to HDL. The binding rates can serve as an indicator of the functional status of HDL.

In yet another embodiment, the invention relates to a method for measuring a binding rate of a biomolecule to high density lipoprotein (HDL) comprising: (a) providing a biosensor with two reflecting surfaces, wherein interference between a reflected beam from a first reflecting surface, which comprises immobilized HDL, and a reflected beam from a second reflecting surface, which lacks immobilized HDL, varies upon binding of a biomolecule to HDL; (b) exposing said biosensor to a biomolecule; (c) determining whether a change occurs in the interference between the reflected beams, whereby such a change is indicative of binding between the biomolecule and HDL; and (d) determining the binding rate between the biomolecule and HDL.

In yet another embodiment, the invention relates to a method for quantifying HDL interactions comprising: (a) providing a biosensor with a first surface comprising biotin-labeled ApoA-1 antibodies and a second surface lacking said antibodies; (b) incubating said biosensor with a sample comprising HDL to produce immunocaptured HDL; (c) incubating said immunocaptured HDL with a solution of a biomolecule, associated with different aspects of HDL metabolism; and (d) determining the rate of association between HDL and said biomolecule.

An advantage of the disclosure is a method that can identify functional HDL that interacts with native biomolecules in an efficient manner.

An advantage of the disclosure is a method that can identify dysfunctional HDL that interacts with biomolecules in an altered fashion.

Advantages of the methods disclosed herein are bioassays of HDL function that can be used to predict atherosclerosis more accurately than HDL cholesterol levels.

An advantage of the methods disclosed herein is an assay of “HDL function” that is well standardized, validated, robust and practical. The assay can be used in clinical laboratories.

An advantage of the disclosure is a method that reveals how oxidation influences HDL interactions with biomolecules that mediate HDL metabolism.

An advantage of the disclosure is a method that can characterize a subject's HDL in a time frame that is compatible with clinical laboratories.

An advantage of the disclosure is a method that uses HDL binding affinity and binding rates for biomolecules as a surrogate for HDL function.

An advantage of the disclosure is a method that can easily (1) identify how composition influences HDL function, (2) show mechanisms that impair HDL function, (3) provide diagnostic benefit to patients and, (4) improve decisions concerning which drugs to use/develop for treating patients who are at risk of heart disease.

An advantage of the disclosure is an in vitro BLI assay that quantifies differences in HDL binding affinity with biomolecules that mediate HDL-dependent cholesterol metabolism.

An advantage of the disclosure is a BLI assay that will provide pathophysiologically relevant data on how disease impacts HDL function and show how disease, drugs and protective mechanisms mediate HDL function, which should improve drug development.

An advantage of the disclosure is a method of HDL function that can be used as clinical assays to predict the patients that are at increased risk of heart disease.

An advantage of the disclosure is novel chimeric biomolecules to assess HDL function.

An advantage of the disclosure is methods that use native binding ligands to determine HDL function.

An advantage of the disclosure is methods that require no labeling, can be performed in a short time period, and that are highly reproducible.

Embodiments of the invention also relate to kits for preparing the methods and compositions of the invention. The kit can be used for, among other things, determining a binding rate of a biomolecule to HDL.

### BRIEF DESCRIPTION OF THE DRAWINGS

FIG. 1 is a diagram depicting the key steps and biomolecules in the reverse cholesterol transport pathway.

FIG. 2A is a schematic depicting biolayer interferometry. BLI is an optical analytical technique that analyzes the interference pattern of white light reflected from two surfaces: a layer of immobilized protein on the biosensor tip, and an internal reference layer. FIG. 2A was obtained from the web site of ForteBio, Inc. (Menlo Park, Calif.)

FIG. 2B is a schematic depicting BLI and showing that any change in the number of molecules bound to the biosensor tip causes a shift in the interference pattern that can be measured in real-time. FIG. 2B was obtained from the web site of ForteBio, Inc. (Menlo Park, Calif.)

FIG. 2C is a schematic depicting BLI and showing that the binding between a ligand immobilized on the biosensor tip surface and an analyte in solution produces an increase in optical thickness at the biosensor tip, which results in a wavelength shift, Δλ, which is a direct measure of the change in thickness of the biological layer. FIG. 2C was obtained from the web site of ForteBio, Inc. (Menlo Park, Calif.)

FIG. 3 is a schematic depicting the use of BLI to detect biomolecules that interact with HDL and to determine binding rates.

FIG. 4A is a line graph demonstrating the loading of biotin-labeled anti-ApoA-I on to a SA biosensor.

FIG. 4B is a line graph showing binding rates of increasing HDL concentrations to the anti-ApoA-I SA bio-sensor.

FIG. 4C is a line graph showing a standard curve of HDL constructed from BLI data in FIG. 4B.

FIG. 5 is a bar graph showing that HOCl oxidation of HDL does not impair rates of binding to anti-ApoA-I biosensors.

FIG. 6 is a bar graph showing the effects of HOCl on HDL affinity for PON1.

FIG. 7 is a bar graph showing the effects of HOCl on HDL affinity for MPO.

FIG. 8 is a bar graph showing the effects of HOCl on HDL affinity for CETP.

FIG. 9 is a bar graph showing the effects of HOCl on HDL affinity for chimeric camelid SR-B 1 (aa. 424-434 in SRB1).

FIG. 10A is a bar graph showing binding rates of HDL in C57B1/6J and Ldlr^{-/-} mice fed western diet (6-8 wk) typical plasma cholesterol=800-1000 mg/dL. HDL binding rates for chimeric SR-B1 (aa424-434) camelid protein are shown (n=4,**=p<0.01).

FIG. 10B is a bar graph showing binding rates of HDL in C57B1/6J and Ldlr^{-/-} mice fed western diet (6-8 wk) typical plasma cholesterol=800-1000 mg/dL. HDL binding rates for MPO are shown (n=4, *=p<0.05).

FIG. 11A is a bar graph showing MPO binding rates for HDL isolated from control hamsters fed chow or high-fructose diets for 3 weeks. The high-fructose diet is a standard rodent model of diet-induced metabolic syndrome. Data are presented as mean+/−SEM (6 animals per group).

FIG. 11B is a bar graph showing the MPO binding rates for HDL isolated from apparently healthy control subjects and patients with physician diagnosed cardiovascular disease. Data are presented as mean+/−SEM (n=6 per group, **=p<0.004). Data are normalized to HDL plasma concentrations.

### DETAILED DESCRIPTION

Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, melt index, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

As used herein, “a” or “an” can mean multiples. For example, “a cell” can mean at least one cell or more than one cell.

The term “antibody,” as used herein, is intended to refer to immunoglobulin molecules comprising four polypeptide chains, two heavy (H) chains and two light (L) chains interconnected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hyper-variability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as diabodies, triabodies, tetrabodies and minibodies, are also encompassed within the expression “antigen-binding fragment,” as used herein.

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a VH domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody using routine techniques available in the art.

As used herein, “ATP-binding cassette transporter” (ABCA1, (member 1 of human transporter sub-family ABCA), also known as the cholesterol efflux regulatory protein (CERP) is a protein that in humans is encoded by the ABCA1 gene. This transporter is a major regulator of cellular cholesterol and phospholipid homeostasis. The membrane-associated protein encoded by this gene is a member of the superfamily of ATP-binding cassette (ABC) transporters. ABC proteins transport various molecules across extra- and intracellular membranes. ABC genes are divided into seven distinct subfamilies (ABCA, MDR/TAP, MRP, ALD, OABP, GCN20, White). Members of the ABCA subfamily comprise the only major ABC subfamily found exclusively in multicellular eukaryotes. With cholesterol as its substrate, this protein functions as a cholesterol efflux pump in the cellular lipid removal pathway.

ABCA1 mediates the efflux of cholesterol and phospholipids to lipid-poor apolipoproteins (apo-A1and apoE), which then form nascent high-density lipoproteins (HDL). It also mediates the transport of lipids between Golgi and cell membrane. Since this protein is needed throughout the body it is expressed ubiquitously as a 220-kDa protein. It is present in higher quantities in tissues that shuttle or are involved in the turnover of lipids such as the liver, the small intestine and adipose tissue.

Factors that act upon the ABCA1 transporter's expression or its posttranslational modification are also molecules that are involved in its subsequent function like fatty acids, cholesterol and also cytokines and cyclic adenosine monophosphate.

As used herein, “ATP-binding cassette sub-family G member 1” is a protein that in humans is encoded by the ABCG1 gene. It is a homolog of the well-known Drosophila gene white.

The protein encoded by this gene is a member of the superfamily of ATP-binding cassette (ABC) transporters. This protein is a member of the White subfamily. It is involved in macrophage cholesterol and phospholipids transport, and may regulate cellular lipid homeostasis in other cell types. Several alternative splice variants have been identified.

As used herein, “Apolipoprotein A-I” is a protein that in humans is encoded by the APOA1 gene. It has a specific role in lipid metabolism. Apolipoprotein A-I is the major protein component of high density lipoprotein (HDL) in plasma. Chylomicrons secreted from the intestinal enterocyte also contain ApoA-I but it is quickly transferred to HDL in the bloodstream. The protein promotes cholesterol efflux from tissues to the liver for excretion. It is a cofactor for lecithin cholesterolacyltransferase (LCAT), which is responsible for the formation of most plasma cholesteryl esters. ApoA-I was also isolated as a prostacyclin (PGI2) stabilizing factor, and thus may have an anticlotting effect. Defects in the gene encoding it are associated with HDL deficiencies, including Tangier disease, and with systemic non-neuropathic amyloidosis.

As used herein, the term “biomolecule” means any molecule that is produced by a living organism, including large polymeric molecules such as proteins, polysaccharides, lipids, and nucleic acids as well as small molecules such as primary metabolites, secondary metabolites, and natural products. The term biomolecule includes recombinant molecules produced by bacteria, viruses, plants and animals.

As used herein the term “biological sample” means an organic material. Some examples of biological samples are proteins, blood, plasma and tissue samples.

As used herein, a “camelid antibody” is an antibody composed of heavy chains only. The camelid antibody was originally found in Camelidae sp. family animals such as Camel and Llama. The variable domain of heavy chain is the smallest single domain antigen-binding fragment. The ˜15 kD fragment, termed “nanobody” can recognize specific antigens extremely well and bind very tightly. Combined with its small size and ease for production in E. coli, the single domain camelid antibody fragment can be used in live cell imaging as well as target molecule isolation.

As used herein, “cell” or “cells,” unless specifically limited to the contrary, includes any somatic cell, embryonic stem (ES) cell, adult stem cell, an organ specific stem cell, nuclear transfer (NT) units, and stem-like cells. The cell or cells can be obtained from any organ or tissue. The cell or cells can be human or other animal. For example, a cell can be mouse, guinea pig, rat, cattle, horses, pigs, sheep, goats, etc. A cell also can be from non-human primates.

As used herein, “cholesteryl ester transfer protein” (CETP), also called plasma lipid transfer protein, is a plasma protein that facilitates the transport of cholesteryl esters and triglycerides between the lipoproteins. It collects triglycerides from very-low-density (VLDL) or low-density lipoproteins (LDL) and exchanges them for cholesteryl esters from high-density lipoproteins (HDL), and vice versa. Most of the time, however, CETP does a homoexchange, trading a triglyceride for a triglyceride or a cholesteryl ester for a cholesteryl ester.

As used herein, “culture medium” or “growth medium” means a suitable medium capable of supporting growth of cells.

As used herein, “glutathione peroxidase 1” is an enzyme that in humans is encoded by the GPX1 gene. This gene encodes a member of the glutathione peroxidase family, consisting of eight known glutathione peroxidases (Gpx1-8) in humans. Mammalian Gpx1 (this gene), Gpx2, Gpx3, and Gpx4 have been shown to be selenium-containing enzymes, whereas Gpx6 is a selenoprotein in humans with cysteine-containing homologues in rodents. In selenoproteins, the 21st amino acid selenocysteine is inserted in the nascent polypeptide chain during the process of translational recoding of the UGA stop codon.

Glutathione peroxidase functions in the detoxification of hydrogen peroxide, and is one of the most important antioxidant enzymes in humans. It has been reported that the protein encoded by this gene protects from CD95-induced apoptosis in cultured breast cancer cells and inhibits 5-lipoxygenase in blood cells, and its overexpression delays endothelial cell growth and increases resistance to toxic challenges. This protein is one of only a few proteins known in higher vertebrates to contain selenocysteine, which occurs at the active site of glutathione peroxidase and is coded by the nonsense (stop) codon TGA. In addition, this protein is characterized in a polyalanine sequence polymorphism in the N-terminal region, which includes three alleles with five, six or seven alanine (ALA) repeats in this sequence. The allele with five ALA repeats is significantly associated with breast cancer risk. Two alternatively spliced transcript variants encoding distinct isoforms have been found for this gene.

As used herein, “hypocholorous acid” (HOCL) is a weak acid with the chemical formula HClO. It forms when chlorine dissolves in water. It cannot be isolated in pure form due to rapid equilibration with its precursor (see below). HClO is an oxidizer, and as its sodium salt sodium hypochlorite, (NaClO), or its calcium salt calcium hypochlorite, (Ca(CIO)₂) is used as a bleach, a deodorant, and a disinfectant.

As used herein, “lecithin-cholesterol acyltransferase” (LCAT, also called phosphatidylcholine-sterol 0-acyltransferase) is an enzyme that converts free cholesterol into cholesteryl ester (a more hydrophobic form of cholesterol), which is then sequestered into the core of a lipoprotein particle, eventually making the newly synthesized HDL spherical and forcing the reaction to become unidirectional since the particles are removed from the surface. The enzyme is bound to high-density lipoproteins (HDLs) and low-density lipoproteins in the blood plasma.

As used herein, “myeloperoxidase” (MPO) is a peroxidase enzyme (EC 1.11.1.7) most abundantly present in neutrophil granulocytes (a subtype of white blood cells). It is a lysosomal protein stored in azurophilic granules of the neutrophil. MPO has a heme pigment, which causes its green color in secretions rich in neutrophils, such as pus and some forms of mucus.

MPO produces hypochlorous acid (HOCl) from hydrogen peroxide (H₂O₂) and chloride anion (Cl⁻) (or the equivalent from a non-chlorine halide) during the neutrophil's respiratory burst. It requires heme as a cofactor. Furthermore, it oxidizes tyrosine to tyrosyl radical using hydrogen peroxide as an oxidizing agent. Hypochlorous acid and tyrosyl radical are cytotoxic, so they are used by the neutrophil to kill bacteria and other pathogens.

As used herein, “platelet activating factor acetylhydrolase” (PAF-AH) is a secreted protein that mediates PAF activity by specifically catalyzing hydrolysis of the “sn2” ester bond, resulting in the conversion of PAF to the biologically inactive lyso-PAF. PAF-AH can also interact with LDL particles to induce the hydrolysis of LDL associated, oxidized phospholipids, generating lysophosphatidylcholine (lyso-PC) and other lysophospholipids. Platelet-activating factor (PAF) is a biologically active phospholipid synthesized by a variety of cells upon stimulation. PAF is converted to the biologically inactive lyso-PAF by the enzyme PAF acetylhydrolase (PAF-AH). Plasma PAF-AH is highly selective for phospholipids with very short acyl groups at the sn-2 position and is associated with lipoproteins.

As used herein, “paraoxonase” (PON1) protein has esterase activity. PON1 is synthesized in the liver and transported along with HDL in the plasma. It functions as an antioxidant; it prevents the oxidation of LDL. Its serum concentration is influenced by inflammatory changes and the levels of serum oxidized-LDL.

PON1 is also a major anti-atherosclerotic component of high-density lipoprotein (HDL). The PON1 gene is activated by PPAR-γ, which increases synthesis and release of paraoxonase 1 enzyme from the liver, reducing atherosclerosis.

As used herein, “peptide,” “protein,” and “polypeptide” mean any chain of amino acids, regardless of length or post-translational modification (e.g., glycosylation or phosphorylation). Unless otherwise specified, the terms are interchangeable. The nucleic acid molecules of the invention encode a variant (mutant) of a naturally-occurring (wild-type) protein or fragment thereof which has substantially the same activity as the full length mutant protein. Preferably, such a mutant protein has an amino acid sequence that is at least 85%, preferably 90%, and most preferably 95% or 99%, identical to the amino acid sequence of a corresponding wild-type protein.

Polypeptide molecules are said to have an “amino terminus” (N-terminus) and a “carboxy terminus” (C-terminus) because peptide linkages occur between the backbone amino group of a first amino acid residue and the backbone carboxyl group of a second amino acid residue. The terms “N-terminal” and “C-terminal” in reference to polypeptide sequences refer to regions of polypeptides including portions of the N-terminal and C-terminal regions of the polypeptide, respectively. A sequence that includes a portion of the N-terminal region of polypeptide includes amino acids predominantly from the N-terminal half of the polypeptide chain, but is not limited to such sequences. For example, an N-terminal sequence may include an interior portion of the polypeptide sequence including bases from both the N-terminal and C-terminal halves of the polypeptide. The same applies to C-terminal regions. N-terminal and C-terminal regions may, but need not, include the amino acid defining the ultimate N-terminus and C-terminus of the polypeptide, respectively.

As used herein, “reverse cholesterol transport” is a multi-step process resulting in the net movement of cholesterol from peripheral tissues back to the liver via the plasma. Cholesterol from non-hepatic peripheral tissues is transferred to HDL by the ABCA1. ApoA-1 acts as an acceptor, and the phospholipid component of HDL acts as a sink for the mobilized cholesterol. The cholesterol is converted to cholesteryl esters by the enzyme LCAT (lecithin-cholesterol acyltransferase). The cholesteryl esters can be transferred to other lipoproteins (such as LDL), and these lipoproteins can be taken up by the liver through its LDL receptors.

However, the receptor SR-B 1 (scavenger receptor class B 1) present on the liver cells' plasma membranes mediates most of the liver's uptake of cholesteryl esters from HDL in the absence of uptake of apolipoproteins. The overall process by which HDL removes cholesterol from extrahepatic tissues and returns it to the liver is called reverse cholesterol transport. Once in the liver, the cholesteryl esters are converted to cholesterol and enter the general pool. Therefore, the liver can eliminate cholesterol from the body by secreting unesterified cholesterol into the bile or by converting cholesterol to bile acids.

Uptake of HDL₂ is mediated by hepatic lipase, a special form of lipoprotein lipase found only in the liver. Hepatic lipase activity is increased by androgens and decreased by estrogens, which may account for higher concentrations of HDL₂ in women.

As used herein, “scavenger receptor class B member 1” (SRB1) also known as SR-BI is a protein that in humans is encoded by the SCARB1 gene. SR-BI functions as a receptor for high-density lipoprotein. Scavenger receptor class B, type I (SR-BI) is an integral membrane protein found in numerous cell types/tissues, including the liver and adrenal. It is best known for its role in facilitating the uptake of cholesteryl esters from high-density lipoproteins in the liver. This process drives the movement of cholesterol from peripheral tissues towards the liver for excretion. This movement of cholesterol is known as reverse cholesterol transport and is a protective mechanism against the development of atherosclerosis, which is the principal cause of heart disease and stroke.

The disclosure relates to methods, compositions and kits to detect HDL interactions with biomolecules. The disclosure relates to methods, compositions and kits to determine a binding rate between HDL and a biomolecule. The methods, compositions and kits can be used to detect dysfunctional or altered HDL. The methods compositions and kits can be used to determine the oxidation state of HDL. In another embodiment, the methods, compositions, and kits can be used to screen for molecules that interact with HDL. In yet another embodiment, the methods, compositions, and kits can be used to screen for drugs and other therapeutic agents.

High Density Lipoprotein

High-Density Lipoprotein (HDL) is the smallest of the lipoprotein particles. They are the densest because they contain the highest proportion of protein. Their most abundant apolipoproteins are ApoA-I and ApoA-II. The liver synthesizes these lipoproteins as complexes of apolipoproteins and phospholipid, which resemble cholesterol-free flattened spherical lipoprotein particles. They are capable of picking up cholesterol, carried internally, from cells by interaction with the ATP-binding cassette transporter A1 (ABCA1). A plasma enzyme called lecithin-cholesterol acyltransferase (LCAT) converts the free cholesterol into cholesteryl ester (a more hydrophobic form of cholesterol), which is then sequestered into the core of the lipoprotein particle, eventually making the newly synthesized HDL spherical. They increase in size as they circulate through the bloodstream and incorporate more cholesterol and phospholipid molecules from cells and other lipoproteins, for example by the interaction with the ABCG1 transporter and the phospholipid transport protein (PLTP).

HDL transports cholesterol mostly to the liver or steroidogenic organs such as adrenals, ovary, and testes by direct and indirect pathways. HDL is removed by HDL receptors such as scavenger receptor BI (SR-BI), which mediate the selective uptake of cholesterol from HDL. In humans, probably the most relevant pathway is the indirect one, which is mediated by cholesteryl ester transfer protein (CETP). This protein exchanges triglycerides of VLDL against cholesteryl esters of HDL. As the result, VLDLs are processed to LDL, which are removed from the circulation by the LDL receptor pathway. The triglycerides are not stable in HDL, but are degraded by hepatic lipase so that finally small HDL particles are left, which restart the uptake of cholesterol from cells.

Biomolecules that Interact with HDL

Biomolecules may interact with HDL directly or indirectly. Biomolecules may interact with HDL independently or as part of a complex. The complex can be composed of any number of proteins including but not limited to 2, 3, 4, 5, 6, 7, 8, 9, 10, 10-20, 21-30, 31-40, 41-50, and greater than 50 proteins.

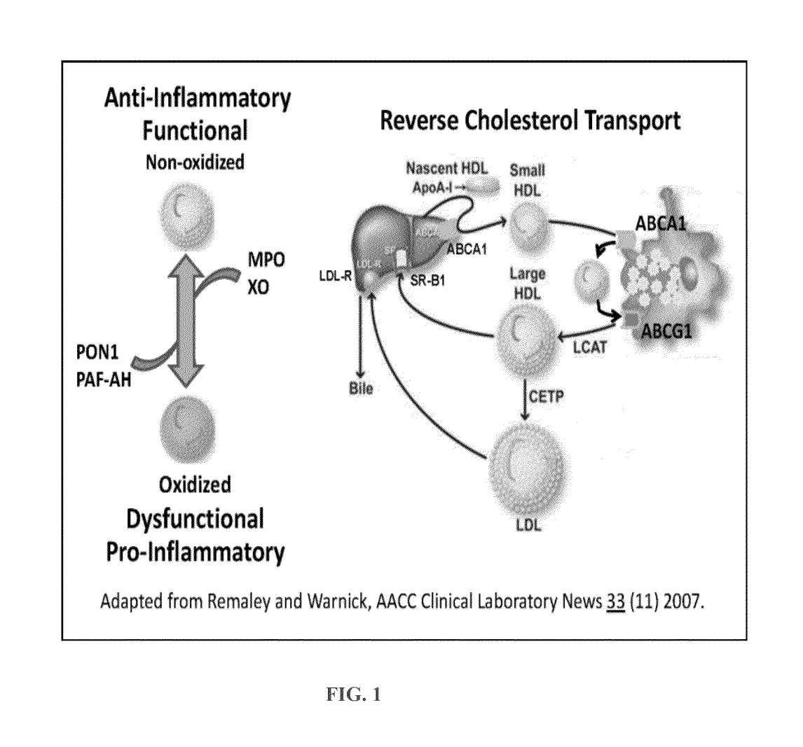

HDL plays an important role in lipoprotein metabolism and also possesses anti-atherosclerotic and anti-inflammatory properties that are difficult to assess. HDL binds a diverse array of cargo made up of lipids and proteins (native as well as oxidized) that influence its ability to bind to its interactome and participate in cholesterol metabolism and transport. For example, lipid-poor HDL binds to the ATP binding cassette transporter A1 (ABCA1), the first step in reverse cholesterol transport (RCT). However, spherical, lipid-rich HDL delivers cholesterol to the liver via scavenger receptor class B type 1 (SR-B 1), the last step in RCT. FIG. 1 provides a diagram outlining steps and biomolecules in the reverse cholesterol transport (RCT) pathway and identifies several representative proteins known to interact with HDL. Anti-inflammatory properties of HDL appear to be linked to its ability to bind paraoxonase [PON], platelet activating factor acetylhydrolase [PAF-AH], myeloperoxidase [MPO] and xanthine oxidase [XO]. HDL performs many functions, and interacts with many proteins. HDL interacts with the proteins that facilitate cholesterol release, esterification, exchange and uptake.

In addition, HDL interacts with ABCA1, ABCG1, LCAT, SR-B1, CETP as shown in FIG. 1. MPO plays an important role in atherosclerosis. Support for this comes from the fact that 2-chlorohexadecanal (2-CHD) and chlorotyrosine are present in vascular lesions. This is important because these products are only formed in vivo by HOCl, which is generated by MPO. 2-CHD is one of the more abundant breakdown products when HOCl reacts with plasmalogens and is formed when HOCl reacts with HDL. 2-CHD is a potent inhibitor of endothelial nitric oxide synthase (eNOS) activity.

Any molecule that interacts with HDL can be assayed by the methods, compositions and kits disclosed herein. Table I provides a representative list of biomolecules that interact with HDL. Fragments, portions, and binding regions of the proteins enumerated in Table I can be screened by the methods, compositions and kits disclosed herein.

TABLE I
HDL-associated proteins identified using modern proteomic strategies

| Protein | Accession | Protein | Accession |
|---|---|---|---|
| Apo A-I | P02657 | LPL | P06858 |
| Po A-II | P02652 | C-RP | P02741 |
| Apo C-III | P02655 | Ceruloplasmin | P00450 |
| Apo A-IV | P02656 | Serum Amyloid P | P02743 |
| Apo E | P06727 | Complement Factor H | P08603 |
| Apo L-I | P02659 | Kallikrein B1 | P03952 |
| SAA4 | O14791 | Factor VIII-assoc. prot. | P00451 |
| SAA1&2 | P35542 | C1-INH | P05155 |
| Apo M | P02735 | MSF-1 | P09603 |
| Apo D | O95445 | Lymphocyte antigen | P06340 |
| Apo J | P05090 | MGEA 5 | O60502 |
| Pon1 | P10909 | HLA-A protein | P04439 |
| HRP | P27169 | NOTCH1 | P46531 |
| Albumin | P00739 | Siglec 5 | O15389 |
| Apo C-1 | P02768 | CLECSF 1 | Q9P126 |
| α-1-antitrypsin | P02654 | Zinc Finger Protein 1A-1 | Q01101 |
| Serotransferrin | P01009 | LTBP-1 | P22064 |
| Transthyretin | P02787 | LTBP-2 | Q14767 |
| Apo F | P02766 | Gas6 | Q61592 |
| α-1B-glycoprotein | Q13790 | Ryanodine receptor 2 | Q92736 |
| CETP | P04217 | POU 5 domain protein | Q8N7G0 |
| Complement C3 | P11597 | TFPI | P10646 |
| α1-acid | P01024 | Unnamed protein product | gi10435007 |
| glycoprotein 2 | | | |
| α2-HS-glycoprotine | P19652 | Unknown protein | gi12653035 |
| Vit. D Binding | P02765 | Unknown protein | gi12802992 |
| Protein | | | |
| Apo C-IV | P02774 | KIAA1095 | A8HTM6 |
| PLTP | P55056 | KIAA1730 protein | Q9C0D3 |
| Pon3 | P55058 | KIAA0675 gene product | Q86Y13 |
| Fibrinogen | Q15166 | ZNF 356 | Q9ULV3 |
| Apo B-100 | P02671 | dj675G8.1 | gi 11137825 |
| Apo H | P04114 | dj733D15.1 | Q3IN78 |
| Complement C4A | P02749 | TAT-interactive protein | gi 1427566 |
| Complement C9 | P0C0L4 | dj758N20.1 | gi 11493357 |
| Vitronectin | | | |
| α-2 antiplasmin | P02748 | PTP | gi 13645209 |
| α-1-microglobulin | P04004 | IgG | P99007 |
| Inter α Trp. Inhib. | P08697 | dj1057B20.2 | Q9NQG5 |
| Angiotensin | P02760 | Desmocollin | Q08554 |
| Kininogen-1 | Q14624 | Alpha-amylase salivary | P04745 |
| Hemopexin | P01019 | Alpha-2-macroglobulin | P01023 |
| SERPINF1 | P01042 | IDH | P50213 |
| LCAT | P02790 | ACTA | P03996 |
| Complement C4B | P36955 | Glaectin-7 (LEG7) | P47929 |
| Ret. Bind. Prot. 4 | P04180 | Platelet basic protein | P02775 |
| Prenylcystein | P0C0L5 | Prothrombin | P00734 |
| oxidase | | | |
| | P02753 | PAF-AH | P43034 |
| | Q9UHG3 | Apo(a) | P085191 |
| | | CD36 | |

In one embodiment, the HDL interacting biomolecule is in a sample selected from the group consisting of a cellular extract, a protein preparation, a purified protein preparation or a protein slurry.

In another embodiment, biomolecules that interact with HDL include but are not limited to PON1, PAF-AH, GPx1, MPO, XO, LCAT, CETP, SR-B1, ABCA1, ABCG1, ABCG4, CD36, Lox-1 (Lectin-like Oxidized LDL receptor) and fragments thereof. The entire biomolecule may be used or only active portions that bind with HDL may be used. In another embodiment, a protein preparation may include one or more of the following biomolecules PON1, PAF-AH, GPx1, MPO, XO, LCAT, CETP, SR-B1, ABCA1, ABCG1, ABCG4, CD36, Lox-1 (Lectin-like Oxidized LDL receptor) and fragments thereof.

The methods, compositions and kits can be used to screen for novel proteins that interact with HDL. Any sample can be screened including a biological sample, a cellular extract, a recombinant protein, a recombinant protein preparation, a slurry, an isolated protein, and a protein preparation.

Biological Sample

In one embodiment, the biological sample is a cell, fetal cell, tissue, blood, serum, plasma, saliva, urine, tear, vaginal secretion, sweat, umbilical cord blood, chorionic amniotic fluid, embryonic tissue, lymph fluid, cerebrospinal fluid, mucosa secretion, peritoneal fluid, ascitic fluid, cellular extract, protein preparation, recombinant protein preparation, fecal matter, or body exudates.

In one embodiment, the biological sample is selected from the group consisting of blood, serum, plasma, lymph fluid, cerebrospinal fluid, peritoneal fluid, and cellular extract.

In one embodiment, the biological sample is obtained from human, non-human, mammal, reptile, cattle, cat, dog, goat, swine, pig, monkey, ape, gorilla, bull, cow, bear, horse, sheep, poultry, mouse, rat, fish, dolphin, whale, or shark. In an embodiment, the biological sample is obtained from a human source.

Detection of Interactions and Determination of Binding Rates

In accordance with the disclosure, HDL interactions can be detected and binding rates can be determined using any number of binding assays including but not limited to in vitro assays, co-immunoprecipitation, fluorescence resonance energy transfer (FRET), bioluminescence resonance energy transfer (BRET), interferometry, biolayer interferometry (BLI), dual-polarization interferometry, Ellipsometry, optical platforms, label free binding assays, Surface Plasmon Resonance, and Quartz Crystal Microbalance.

In one embodiment, optical technique may be used. In still another embodiment, “label-free,” optical techniques may be used meaning that it does not require the attachment of a radioactive, fluorescent, or other label to the HDL molecules. Table II provides a representative list of label-free systems that can be used. Any of the systems provided in Table II can be used to determine a binding rate between HDL and a biomolecule.

TABLE II
A representative list of label-free systems for binding analysis

| | | BIOSENSOR | | BINDING ANALYSIS APPLICATION |
|---|---|---|---|---|
| VENDOR | SYSTEM NAME | TECHNOLOGY | FORMATS | FOCUS |
| Attana | A100 ® and | Quartz | Chip-based flow cells | Antibody selection and characterisation, |
| | A100 ® C-fast | Crystal | | crude samples, detailed kenetic analysis, |
| | with | Microbalance - | | selectivity profiling, epitope binding, |
| | autosampler | QCM | | concentration measurements |
| Bio-Rad | ProteOn | SPR | 6 × 6 Microchannel | Small molecule affinity screening and |
| Laboratories | XPR36 | | flow cell array | validation, antibody profiling and |
| | | | | characterization, phage display Fab |
| | | | | panning, kinetic analysis |
| Corning | Epic ® System | Optical | 384-well plate | Yes/No binding, affinity determination, |
| | | Grating | | direct bind, functional assays, fragment- |
| | | | | based screens, protein/protein |
| | | | | interactions |
| ForteBio | Octet ® RED | BioLayer | Optical biosensor | Small molecule binding and affinity, |
| | | Interferometry | tips, provided in a | fragment screening, small peptide and |
| | | | 96-well plate, | antibody characterisation, and protein |
| | | | fluidics-free | quantitation |
| Fujifilm | AP-3000 | SPR | Samples loaded | Fragment screening, secondary |
| | | | automatically in 384- | screening, in silico derived compound |
| | | | well plate, analysed | screening, small molecule affinity and |
| | | | in disposable sensor | kinetic analysis |
| | | | stick | |
| GE Healthcare | Biacore ™ T100 | SPR | Chip-based flow cells | Antibody characterisation, small |
| | | | | molecule affinity/kinetic analysis, |
| | | | | selectivity profiling, focused libraries and |
| | | | | fragment screening, immunogenicity |
| | | | | studies |
| Graffinity | PlasmonImager ™ | SPR Imaging | Array-based | Small molecule affinity analysis, |
| Pharmaceuticals | | | chemical | fragment-based screening, selectivity |
| | | | microarrays, up to | profiling, binding mode profiling |
| | | | 9,126 spots per array | |
| GWC | SPRimager ® II | SPR Imaging | Flexible array | Biomolecular interactions for proteins, |
| Technologies | | | formats, manual or | peptides, antibodies, nucleic acids, |
| | | | robotic spotting | carbohydrates, whole cells, biosensor |
| | | | | and bioarray development |
| ICx | SensiQ | SPR | Chip-based flow cells | Antibody screening and selection, small |
| Technologies | | | | molecule affinity and kinetics, protein |
| | | | | interaction characterisation |
| MicroCal | Auto-iTC₂₀₀ ™ | Isothermal | 200 μL sample cell, | Mechanism of action studies, assay |
| | | Titration | fully automated, 75 | validation, hit validation, fragment |
| | | Calorimetry | samples/day | screening, lead optimization, protein |
| | | | | characterisation |
| Reichert | SR 7000 Series | SPR | Chip-based flow cells | Molecular interaction characterization |
| Analytical | | | | (kenetics, affinity, equilibrium); SPR |
| Instruments | | | | with electrochemistry, photochemistry, |
| | | | | fluorescence via custom flowcells |
| Silicon Kinetics | SKi Pro | nPOI | Differential flow cell, | Yes/No, affinity and kinetic |
| | | (Nanopore | 96-well sensor plate | characterizations of biomolecular |
| | | Optical | | interactions, viral cross hybridisation |
| | | Interferometry) | | |
| SRU | BIND ® | Photonic | 96-, 384- and 1536- | Antibody screening, epitope analysis, |
| Biosystems | | Crystal | well plates, 8- and | small molecule and fragment screening, |
| | | | 16-well cartridges | selectivity profiling, stoichiometry and |
| | | | | mechanism of binding |
| TA | Nano ITC^{2G} and | Isothermal | Nano ITC^{2G} - 1.0 mL | Full thermodynamic characterization of |
| Instruments | TAM III | Titration | fixed-in-place | protein-protein interactions, drug-target |
| | | Calorimetry | stirred cell; TAM III - | binding, structure/function relationships |
| | | | I, 4, 20 mL | and production lot binding activity |
| | | | removable stirred | validation |
| | | | cells. All testing | |
| | | | done in solution | |
| TTP Lab Tech | RAPid 4 | Resonant | Sensor cassettes | Quantification and kinetic analysis of |
| | | Acoustic | containing 2 flow | protein-protein interactions, antibody |
| | | Profiling ™ | cells | optimsation, protein expression analysis, |
| | | (RAP) | | quality control |

In some embodiments, the label-free optical method that is employed can detect changes upon exposure to the sample in optical and/or physical properties of a film. Illustrative of label-free techniques are interferometry, surface plasmon resonance, and ellipsometry.

1. In Vitro Assays

A convenient assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of an in vitro, cell free assay is a binding assay. While not directly addressing function, the ability of a modulator to bind to a target molecule in a specific fashion or to inhibit the binding of other molecules is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determination of binding. Usually, the target will be the labeled species, decreasing the chance that the labeling will interfere with or enhance binding. Of particular interest here are competitive binding formats that can be performed in which one of the agents is labeled, and one may measure the amount of free label versus bound label to determine the effect on binding. Assays for assessing binding rates include gel mobility shift, FRET, BRET, protein pull-down, or ELISA based detection of complex formation.

2. Activity Assay

Another example of an in vitro assay is an activity assay. Enzymes catalyze reactions, converting of substrate molecules to products. Direct interactions between the substrates and the specific enzyme of a particular reaction facilitate the alterations that result in product formation. Enzymes may be targeted by molecules that inhibit or enhance their activity. Direct enzyme inhibition is the subject of many successful drug screens. In many cases the regulation of an enzyme is accomplished through protein-protein interactions. Thus the regulation of enzyme activity and complex formation are equivocal. Such regulatory mechanisms serve as important specific targets for therapeutic drug development. In such cases, an activity assay must include the detection of the effect of the activating or inhibiting protein and then the ability of candidate substance to interfere with that regulatory effect. An assay designed to detect regulation of enzyme activity and distribution thereof relies upon detection of enzyme activity as reflected in the disappearance of substrate or the accumulation of product. Methods include colorimetric, fluorescent, or radionuclide detection. Substrates or products may be directly detected or indirectly quantitated via immunodetection or some other secondary enzymatic measure. Permutations of enzymatic assays are widely known and numerous.

3. Co-Immunoprecipitation

Protein-protein interactions may also be studied by using biochemical techniques such as cross-linking, co-immunoprecipitation, co-fractionation by chromatography, and “pull-down” assays, which are well known to those skilled in the art. The co-immunoprecipitation technique consists of (i) generating a cell lysate; (ii) adding an antibody to the cell lysate; (iii) precipitating and washing the antigen; and (iv) eluting and analyzing the bound proteins. The antigen used to generate the antibody can be a purified protein, or a synthetic peptide coupled to a carrier. Both monoclonal and polyclonal antibodies can be utilized in co-immunoprecipitation, or alternatively, a protein can be used which carries an epitope tag recognized by a commercially available antibody. An in vitro correlate pull-down assay may also be used where the purified protein is immobilized on a resin via covalent crosslinking or through affinity interactions with a moiety presented by the resin (e.g., nickel-NTA). Ligand protein may be added to the immobilized protein and binding may be monitored via immunodetection, or scintillation counting if the ligand is radiolabeled.

4. Fluorescence Energy Transfer (FRET)

FRET is a phenomenon in which the excited-state energy in one molecule (called the donor) is transferred to another molecule by a radiationless coupling. This mechanism was first correctly described by Forster, and differs from other types of energy transfer, such as electron sharing (Dexter) or trivial transfer (emission of a photon from the donor and reabsorption by the acceptor). The Dexter mechanism requires the two molecules to be in physical contact, while trivial transfer is a very low probability. In contrast, the Forster mechanism exhibits a high probability when the two molecules are within the Forster radius, which is defined for any given pair of fluorophores.

The overall FRET efficiency depends on the Forster radius, and is determined by several factors and is directly related to the amount of overlap between the absorption spectra of the acceptor molecule and the emission spectra of the donor molecule. The amount of FRET also depends on the alignment of the donor and acceptor molecules, although most biological systems are not rigidly aligned. The FRET efficiency is also affected by the ability of the acceptor molecule to absorb light, as indicated by its molar extinction coefficient, and the overall stability of the excited state of the donor molecule, as indicated by the probability that absorption will lead to fluorescence (quantum yield) and the lifetime of the excited state.

FRET between two different fluorophores can be assayed by several methods: looking at the change in color of the fluorescence, measuring the fluorescence lifetime of the donor, examining the changes upon photobleaching either the donor or acceptor, or as we show in this new invention: by measuring the fluorescence polarization of the acceptor. Regardless of the approach, most of these assays share common features of the instrumentation.

5. BRET

An adaptiation of FRET is bioluminescence resonance energy transfer or BRET. BRET utilizes an enhanced variant of YFP, citrine, and Renilla luciferase to reveal strong resonance energy transfer from the active luciferaase to the YFP, thereby invoking invoke epifluourescence. The advantage of BRET over FRET is that exogenous excitation is not required and multiple cells can be used to derive the binding data, although the emission energy is substantially less than FRET.

6. Interferometry

Interferometry is a technique based on measurement of a light intensity produced by an interference of two or more light beams. Interferometry can be used for detecting optical properties, such as a refraction index, and physical properties, such as thickness, of a thin film when a difference between the light beams is due to the light passing through the thin film. Experimental configurations for interferometry include but are not limited to reflection and transmission configuration. For example, for a thin film disposed on a substrate, two interfering beams in the reflection mode can be (1) a beam passing through the thin film and reflecting from an interface between the substrate and the film and (2) a beam reflecting from an interface between the thin film and the air. If the substrate is optically transparent, an interference can be measured in a transmission mode as well. Two interfering beams in this case can be (i) a beam passing through the thin film and the substrate without any reflections and (ii) a beam passing through the thin film; reflecting at an interface between the thin film and the substrate; reflecting back at the interface between the thin film and the air, and then passing through the substrate.

Interferometry can be used for detecting a change in thickness of an organic film, comprised of HDL molecules, consequent to exposure to a biological sample, and thereby for determining the amount of HDL interacting biomolecules in the sample from the detected change in thickness.

In some embodiments, an interferometric apparatus can be one where an interference is created between light beams passing through two or more identical waveguides. Such waveguides can be waveguides as disclosed, for instance, in U.S. Pat. No. 7,062,110, U.S. Pat. No. 7,050,176, U.S. Pat. No. 6,701,032, and U.S. Pat. No. 6,335,793. In such configuration, one of the waveguides can act as a reference and another of the waveguides can be a test waveguide. Such an interferometric apparatus can measure a change in thickness and optical properties of the thin film disposed on the outer surface of one of waveguides.

For the present application, the waveguide interferometric apparatus can have a thin film, comprised of HDL molecules, that is disposed on one of the waveguides, and a measurement of changes in the thickness of the film upon an exposure to a biological sample can be used for detecting the presence or absence of HDL interacting biomolecules in the sample. The interferometric apparatus with two identical waveguides can function in dual polarization interferometry mode, i.e., a polarizer switching between p- and s-polarization (TE and TM modes) can be placed on a common optical path of two beams between a light source and a detector. Dual polarization interferometry apparatus is available commercially from Farfield Scientific Limited (Cheshire, U.K.).

In some embodiments, an interferometric apparatus for identifying biomolecules that interact with HDL in a sample can be a fiber-optic assay apparatus disclosed in US patent publication No. 2005/0254062. Such an apparatus is available commercially from Fortebio, Inc. (Menlo Park, Calif.). The type of interferometry disclosed in US patent publication No. 2005/0254062 is commercialized by Fortebio as Biolayer Interferometry (BLI).

7. Biolayer Interferometry (BLI)

Biolayer Interferometry (BLI) is a label-free technology for measuring biomolecular interactions. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time.

The binding between a ligand immobilized on the biosensor tip surface and an analyte in solution produces an increase in optical thickness at the biosensor tip, which results in a wavelength shift, Δλ, which is a direct measure of the change in thickness of the biological layer. Interactions are measured in real time, providing the ability to monitor binding specificity, rates of association and dissociation, or concentration, with precision and accuracy.

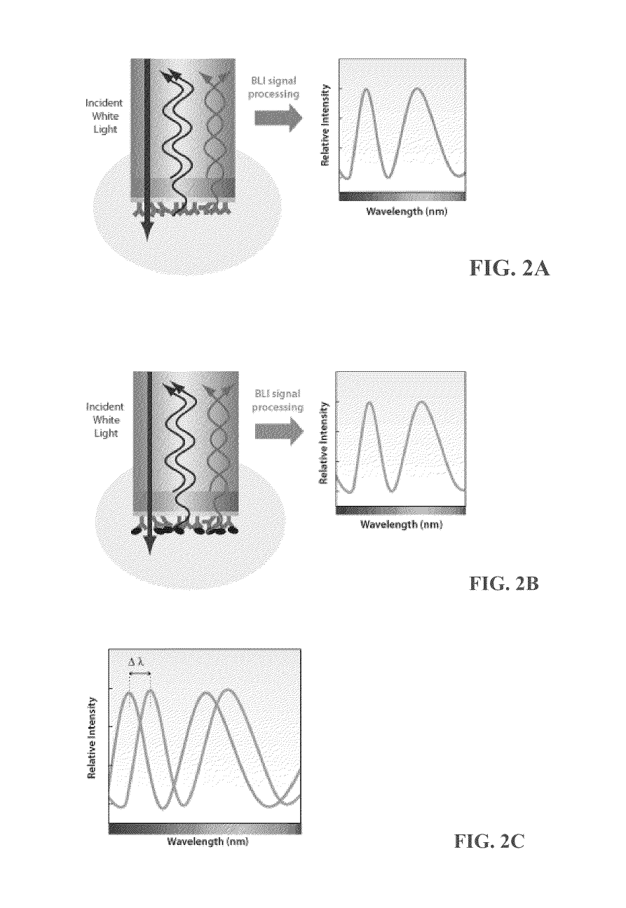

FIG. 2 illustrates principle of operation for BLI. In FIG. 2, interfering light beams originate from 1) the interface with the optical layer (a) and from the surface of the biocompatible layer comprising immobilized molecules (b) where the biocompatible layer meets the surrounding solution. The light beam can interact constructively or destructively as respectively demonstrated by the left two waves and the right two waves (FIG. 2B-2C). Binding of molecules from the solution will change optical properties and/or thickness of the biocompatible layer and will result in changes in the interference pattern.

For the present invention, the biocompatible layer used for BLI can be a film comprising immobilized HDL molecules, and molecules in the solution binding to the biocompatible layer can be HDL interacting biomolecules, as described above. Several methods can be used to immobilize HDL molecule to the biocompatible layer, including the use of an antibody.

A representative protocol for an analytical assay for quantifying HDL interactions with biomolecules is provided below. The representative protocol is for use with the Octet Red system. However, one of ordinary skill in the art will understand how to optimize the protocol for a specific system.

First, a biological sample is obtained. In this example, human plasma is isolated from whole blood drawn into heparin tubes (green top tube). Next, an aliquot of plasma (40 μL is mixed with 160 μL of 1× assay buffer (forteBio, physiological PBS with BSA) mixed and pipetted into a black plastic 96 well plate. A strepavidin-coated biosensor is incubated with biotin-labeled anti-ApoA-I antibodies until the saturation is achieved. Saturation can be determined by watching changes in wavelength interference.

Once saturation of the biosensor is achieved, the antibody coated biosensors are washed free of excess antibody in 1×-assay buffer. Once the washes are completed, the anti-ApoA-I antibody coated biosensor is incubated in the plasma sample for 15-20 minutes. Rates of HDL binding (increases in wavelength interference) to the biosensor are used to calculate HDL concentrations.

Immunocaptured HDL on the biosensor is incubated in lx assay buffer for 3-5 minutes to achieve baseline. Next, the washed immunocaptured HDL is incubated in solutions of biomolecules associated with different aspects of HDL metabolism (see FIG. 1 and Table I for representative examples). Rates of association (increase in wavelength interference) indicate the extent to which the subject's HDL promotes or restricts biomolecular interactions. Changes in wavelength interference can be compared to the changes in wavelength interference for solutions of increasing concentrations of native and oxidized HDL, which yield standard curves.

8. Dual Polarization Interferometry (“DPI”)

DPI measures the structure of a protein by determining its thickness and refractive index. DPI uses orthogonal polarized light from a laser via transverse electric (TE) and transverse magnetic (TM) light waves, passing through a waveguide on an AnaChip™ surface. Variations in the inference pattern are caused by changes in the structure and/or mass of the immobilized molecules. Thus, DPI provides information indicative of structural changes taking place in molecular systems as they function and interact.

9. Ellipsometry

Ellipsometry is a technique that measures a change in polarization that an incident, polarized beam of light experiences upon reflection from a specular surface. Ellipsometry can be used for determining optical properties, such as refraction coefficient, and physical properties, such as thickness, of thin organic films disposed on the specular surface. Principles of ellipsometry and related applications for measuring properties of thin films is detailed, for example, in Collins, R. W. et al., “Spectroscopic Ellipsometry,” in CHARACTERIZATION OF ORGANIC THIN FILMS 35-55 (Butterworth-Heinemann, 1995). Ellipsometry can be used for measuring, upon an exposure to the sample from the subject, a change in thickness and/or optical properties of a thin film comprised of HDL molecules.

Typical ellipsometer includes at least the following components: a light source, a detector, a polarizer positioned on the optical path between the light source and the sample of study; an analyzer, which is a polarizer positioned on the optical path between the sample of study and the detector. Real-time ellipsometers are available commercially from J. A. Woolham Co., Inc., among others.

Ellipsometry can be applied for measuring a change in optical properties, such as refractive index, and/or thickness in a thin organic film that is disposed on the specular surface and contains HDL molecules, upon exposure to a biological sample. From the measured change, a binding rate between HDL and a biomolecule in a sample can be determined.

10. Surface Plasmon Resonance

Surface Plasmon Resonance (SPR) is a technique based on a physical process that can occur when plane-polarized light hits a thin metallic film under total internal reflection condition. SPR can be used for detecting binding events that occur in an organic film deposited on the thin metallic film. In one embodiment, such an organic film can comprise HDL molecules, and the binding events can be those between a biomolecule from the sample and the HDL molecules. Details of SPR can be found, for example, in Biacore AB, TECHNOLOGY HANDBOOK (1998); and Markey, Bio Journal 1: 14-17 (1999). Illustrative SPR instrumentation is commercially available from Biacore AB (Rapsgatan, Sweden).

To detect molecular interactions, one first can obtain an SPR element, which includes a dielectric element and a thin metallic film deposited on the dielectric element; then form a thin organic film containing HDL molecules on a surface of the metallic thin film not facing the dielectric element; then expose the thin organic film to the sample and measure changes in an SPR signal upon the exposure.

The dielectric element can have any appropriate configuration capable to generate and measure surface plasmon resonance. For instance, the dielectric element can be a prism in an Otto or Kretchman configuration; a waveguide or a sinusoidal grating.

The thin metallic film can any a thin film of any metal capable to produce surface Plasmon effect. The thin metallic film can be gold or silver film, for example.

SPR measurement of SPR typically involves a measurement of angular dependence of intensity of p-polarized light reflected from an internal surface of the metallic thin film, i.e., a surface of the metallic thin film facing the dielectric element.

11. Quartz Crystal Microbalance

Non-optical label-free methodology, illustrated by the use of a quartz crystal microbalance (QCM) can also be used to detect HDL interacting biomolecules in a sample. QCM is a technique that measures a mass added to a piezoelectric quartz crystal by detecting a change in the frequency of the crystal. Quartz crystal microbalance systems are available commercially from Stanford Research Systems (Sunnyvale, Calif.).

Pursuant to this approach, one can dispose, on the surface of a piezoelectric crystal, a thin organic film comprised of HDL molecules, and then expose the film to the sample. By measuring whether the exposure occasions a change in the frequency of the piezoelectric crystal, it is possible to ascertain any change in thickness of the film and, as a function of such change, to extrapolate the presence or absence, and amounts, of HDL interacting biomolecules in a sample.

12. Self-Assembled Monolayers

Self assembled monolayers (SAMs) are used to optimized the localized surface plasma resonance (Straker et al, Optimizing the Localized Surface Plasmon Resonance Biosensor through Self-Assembled Monolayers, Nanoscape, Vol. 7, Issue 1, pp. 15-18 (2010).

Self assembled monolayer is an organized layer of amphiphilic molecules in which one end of the molecule, the “head group” shows a specific, reversible affinity for a substrate. SAMs also consist of a tail with a functional group at the terminal end. SAMs are created by the chemisorption of hydrophilic “head groups” onto a substrate from either the vapor or liquid phase⁻followed by a slow two-dimensional organization of hydrophobic “tail groups.” Initially, adsorbate molecules form either a disordered mass of molecules or form a “lying down phase,” and over a period of minutes to hours, begin to form crystalline or semicrystalline structures on the substrate surface. The hydrophilic “head groups” assemble together on the substrate, while the hydrophobic tail groups assemble far from the substrate. Areas of close-packed molecules nucleate and grow until the surface of the substrate is covered in a single monolayer.

Adsorbate molecules adsorb readily because they lower the surface free-energy of the substrate and are stable due to the strong chemisorption of the “head groups.” These bonds create monolayers that are more stable than the physisorbed bonds of Langmuir-Blodgett films.

Thiol-metal bonds, for example, are on the order of 100 kJ/mol, making the bond stable in a wide variety of temperature, solvents, and potentials. The monolayer packs tightly due to van der Waals interactions, thereby reducing its own free energy. The adsorption can be described by the Langmuir adsorption isotherm if lateral interactions are neglected. If they cannot be neglected, the adsorption is better described by the Frumkin isotherm.

SoPRano™ GNR technology platform also can be used to detect HDL interacting biomolecules. SoPRano™ GNR technology enables high throughput, plate-based, homogenous SPR assays using a standard absorbance plate reader, enabling the broader adoption of SPR label-free biomolecular analysis at all stages of basic research and discovery. In essence, it is an adaptation for studying surface Plasmon resonance. It uses a standard UV-VUS scanning plate reader to quantify protein-protein interactions. Equioment is available from PharmaDiagnostics.

HDL Binding Rates with Biomolecules as a Risk Marker for Disease

In one embodiment, the invention relates to a method for determining a binding rate between HDL and a biomolecule in a biological sample obtained from a subject. In one embodiment, the HDL may comprise a mixture of native HDL and oxidized HDL. The binding rate between HDL and a biomolecule in a biological sample from the subject may be compared to a control value, which is derived from measurements between HDL and the biomolecule in comparable biological samples obtained from a population of control subjects. Alternatively, the binding rate between HDL and the biomolecules in a biological sample obtained from the subject may be compared to an internal standard. Examples of such internal standards include, but are not limited to, a binding rate obtained prior to the presence of a negative symptom.

In one embodiment, the comparison characterizes the subject's risk of having cardiovascular disease (CD), as determined using standard protocols for diagnosing CVD. Moreover, the extent of the difference between a subject's binding rate between HDL and a biomolecule and the control value is also useful for characterizing the extent of the risk and thereby determining which subjects would most greatly benefit from certain therapies. In another embodiment, the comparison characterizes the subject's risk of developing CVD in the future. In another embodiment, the comparison can be used to characterize the subject's risk of experiencing a complication of CVD or a major adverse cardiac event, such as myocardial infarction, re-infarction, need for revascularization, stroke, and/or death, within one week to four weeks, one to twelve months, or one to 10 years after the sample is taken. The methods can also be used to determine if a subject presenting with chest pain is at risk of experiencing a major adverse cardiac event, such as a myocardial infarction, re-infarction, need for revascularization, stroke and/or death, near term, e.g., within the following day, 3 months, or 6 months after the subject presents with chest pain.

Also provided herein are methods for monitoring over time the status of CVD in a subject. In one embodiment, the method comprises determining the binding rate between HDL and at least one biomolecule in a biological sample taken from the subject at an initial time and in a corresponding biological sample taken from the subject at a subsequent time. A change in the binding rate between HDL and a biomolecule at the subsequent time as compared to the initial time indicates that a subject's risk of having CVD has changed. For those subjects who have already experienced an acute adverse cardiovascular event such as a myocardial infarction or ischemic stroke, such methods are also useful for assessing the subject's risk of experiencing a subsequent acute adverse cardiovascular event.

In another embodiment, the invention provides a method for characterizing a subject's response to therapy directed at stabilizing or regressing CVD. The method comprises determining a binding rate between HDL and at least one biomolecule in a biological sample taken from the subject prior to therapy and determining the binding rate between HDL and a biomolecule n a corresponding biological sample taken from the subject during or following therapy. A change in the binding rate between HDL and a biomolecule in the sample taken after or during therapy as compared to the binding rate before therapy is indicative of either positive or negative effects of drug therapy.

Kits

Embodiments of the invention also relate to kits for preparing the methods and compositions of the invention. The kit can be used for, among other things, detecting interactions and determining binding rates between HDL and a biomolecule. The kit may comprise at least one antibody that can be used to immobilize HDL. The kit can contain a biosensor to be used in BLI.

The kit may comprise purified HDL or an expression vector that can be used to express and purify HDL. The kit may comprise a bacterium, yeast or virus that can be used to induce expression of HDL. The kit may comprise biomolecules known to interact with HDL including but not limited to PON1, PAF-AH, GPx1, MPO, XO, LCAT, CETP, SR-B1, ABCA1, ABCG1, ABCG4, CD36, Lox-1 (Lectin-like Oxidized LDL receptor) and fragments thereof The reagents of the kit can be provided in a single container or in multiple containers.

The kit may comprise the necessary reagents for an analytical assay to quantify HDL interactions with other biomolecules. The kit may comprise the necessary reagents to determine one or more binding rates between HDL and a biomolecule.

The kit may also contain an instructional material, which describes the use of the components provided in the kit. As used herein, an “instructional material” includes a publication, a recording, a diagram, or any other medium of expression that can be used to communicate the usefulness of the methods of the invention in the kit for, among other things, detecting binding between HDL and a biomolecule. Optionally, or alternately, the instructional material may describe one or more methods of determining a binding rate between HDL and a biomolecule. The instructional material of the kit of the invention may, for example, be affixed to a container, or component thereof. Alternatively, the instructional material may be shipped separately from the container with the reagents with the intention that the instructional material will be used cooperatively by the recipient.

The invention is now described with reference to the following Examples. These Examples are provided for the purpose of illustration only and the invention should in no way be construed as being limited to these Examples, but rather should be construed to encompass any and all variations that become evident as a result of the teaching provided herein. All references including but not limited to cited references, U.S. patents, allowed U.S. patent applications, or published U.S. patent applications are incorporated within this specification by reference in their entirety.

### EXAMPLES

The following examples are illustrative only and are not intended to limit the scope of the invention as defined by the claims.

### Example 1

HDL Binding Affinity for Native Biomolecules can be used as a Surrogate for HDL Function. Methods are disclosed herein, which challenge the current paradigm of patient care, by proposing a new way of quantifying HDL function. Assays that measure interactions between HDL and the biomolecules that are well known to mediate HDL metabolism are disclosed.

HDL binds a wide variety of biomolecules. Some interactions between HDL and biomolecules appear to be influenced by diseases driven by oxidative stress and inflammation. BLI assays were used to measure HDL/ApoA-I concentrations.

BLI assays of HDL interactions with biomolecules represent a novel approach for obtaining biologically relevant information about “HDL function.” If a change in HDL composition is pathophysiologically relevant, then it should alter the way HDL interacts with biomolecules that mediate HDL function.

Methods

BLI is a relatively new, label-free technique for measuring biomolecular interactions. BLI analyzes the interference pattern of light reflected from two surfaces. The first surface is the immobilized protein, which is the internal reference. As the number of biomolecules binding to the biosensor increases, the protein layer increases, causing a shift in interference that can be measured in real-time.

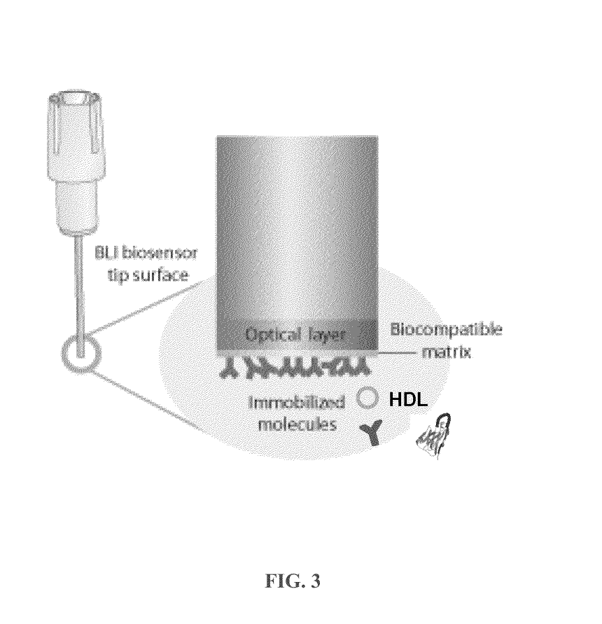

The Octet Red is a fully automated, 8 channel BLI. It requires small sample sizes (<100 μL, 15 nL at the interface), is highly reproducible (<3% CV) and capable of determining concentrations at the sub ng/mL level. The Octet Red can measure protein concentration (via rates of antigen-antibody binding) in 96 samples in <20 min at a cost of ˜30¢/sample. Furthermore, HDL binding affinity (i.e., kₒₙ/k_{off}) can be determined quickly because you can use the same biosensor as was used to quantify ApoA-I. As HDL binding rates can be measured in real time, labeling of protein is not required, thus eliminating a source of interference. A schematic of BLI used with HDL is shown in FIG. 3.

Results

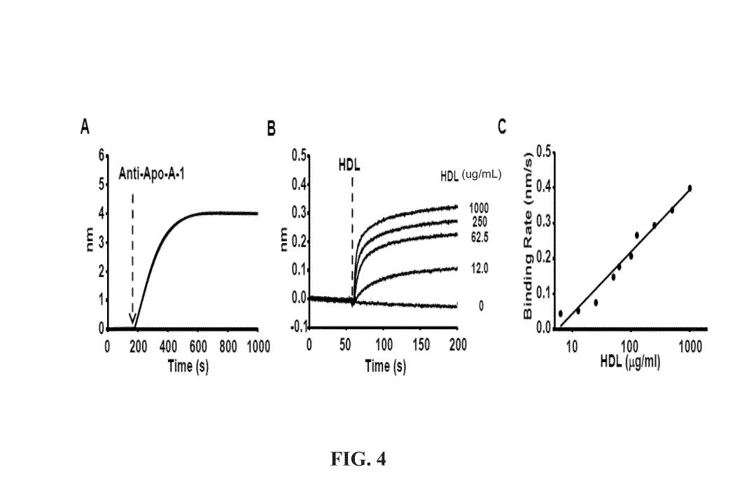

FIG. 4A is a line graph demonstrating an increase in wavelength interference as biotin-labeled anti-ApoA-I antibodies bind to streptavidin (SA)-coated biosensors over time. After saturating the SA-biosensor with biotin-labeled anti-ApoA-I antibody, the biosensors were incubated in increasing concentrations of HDL. FIG. 4B shows that native HDL (n-HDL) rapidly binds to the biosensors in direct relation to concentration. FIG. 4C shows that binding rates, calculated from data in FIG. 4B, yield a standard curve.

### Example 2

As HDL function is hypothesized to decrease with increasing oxidative stress, native HDL was incubated with HOCl under conditions that are reported generate ox-HDL (oxidized HDL), which is similar to that detected in vivo (Zheng L, et al., J Clin Invest. 2004; 114:529-541). The ability of the anti-ApoA-I-biosensors to bind n-HDL and ox-HDL was compared.

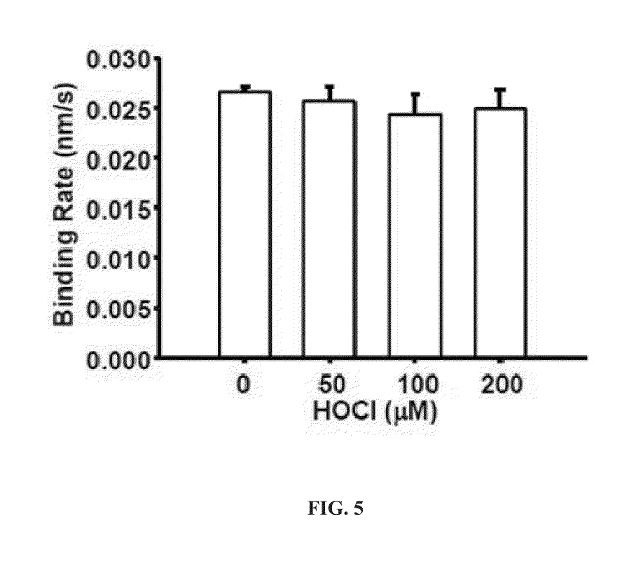

FIG. 5 shows that n-HDL and ox-HDL bind to the anti-ApoA-I biosensor at essentially the same rate. Thus, the biosensors detected ox-HDL as well as n-HDL, which is essential for developing an unbiased assay for measuring differences in binding affinity and/or rates of binding. The data presented in FIGS. 2-5 provide strong proof that biosensors capture n-HDL and ox-HDL equally well to detect structural differences.

### Example 3

After establishing that the ApoA-I biosensor detected and quantified n-HDL and ox-HDL equally well, an investigation was conducted to determine if n-HDL and ox-HDL exhibit any differences in binding affinity for PON1 (15 μg/mL).

Purified human HDL (1 mg protein/mL) was labeled with biotin. Streptavidin (SA) biosensors were incubated for 15 minutes in a standard solution of the biotin-labeled HDL. The HDL-coated SA biosensors were then incubated in 100 μM HOCL in PBS or PBS alone for 15 minutes followed by a PBS wash for 3 minutes. Finally, the non-oxidized and HOCL-oxidized HDL coated SA biosensors were incubated in solutions of PON1, MPO, CETP or the chimer camelid SR-B1 (amino acids 424-434 in SRB1) for 15 minutes and rates of protein binding determined by BLI.

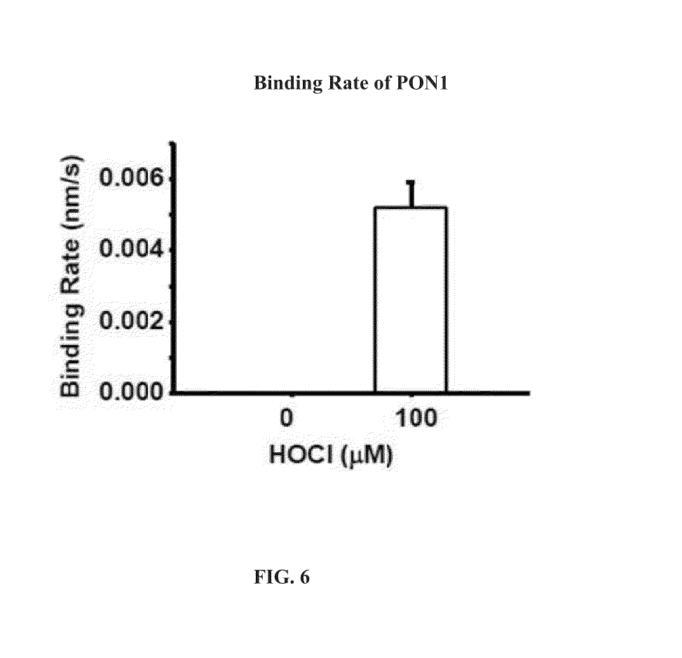

FIG. 6 shows that that PON1 binding rates with ox-HDL were much faster than the rates at which it associates with native HDL, which is zero. These data are consistent with reports that PON1 binds ox-HDL to help protect it from further oxidation resulting from degradation of lipid hydroperoxides. This experiment demonstrates that the methods disclosed herein can be used to detect interactions that occur with ox-HDL. In addition, the methods can be used as an assessment as to the state of functionality of HDL.

### Example 4

Although HDL binds MPO, exactly how oxidation modulates HDL interactions with MPO remains unknown. To determine how oxidation alters HDL binding to MPO, biosensors were loaded with n-HDL or ox-HDL and incubated in BSA-buffers with MPO.

Purified human HDL (1 mg protein/mL) was labeled with biotin. Streptavidin (SA) biosensors were incubated for 15 minutes in a standard solution of the biotin-labeled HDL. The HDL-coated SA biosensors were then incubated in 100 μM HOCL in PBS or PBS alone for 15 minutes followed by a PBS wash for 3 minutes. Finally, the non-oxidized and HOCL-oxidized HDL coated SA biosensors were incubated in solutions of PON1, MPO, CETP or the chimer camelid SR-B1 (amino acids 424-434 in SRB1) for 15 minutes and rates of protein binding determined by BLI.

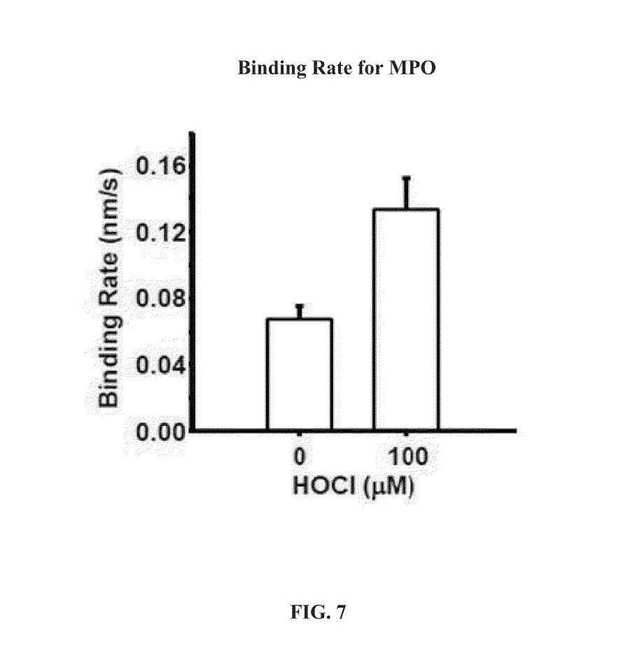

FIG. 7 shows that MPO binding rates for ox-HDL were much faster than for n-HDL. These data show the binding rates between two different forms of HDL (n-HDL and ox-HDL) and a biomolecule can be determined.

### Example 5

n-HDL and ox-HDL binding rates for CETP (15 μg/mL) were compared.

Purified human HDL (1 mg protein/mL) was labeled with biotin. Streptavidin (SA) biosensors were incubated for 15 minutes in a standard solution of the biotin-labeled HDL. The HDL-coated SA biosensors were then incubated in 100 μM HOCL in PBS or PBS alone for 15 minutes followed by a PBS wash for 3 minutes. Finally, the non-oxidized and HOCL-oxidized HDL coated SA biosensors were incubated in solutions of PON1, MPO, CETP or the chimer camelid SR-B 1 (amino acids 424-434 in SRB1) for 15 minutes and rates of protein binding determined by BLI.

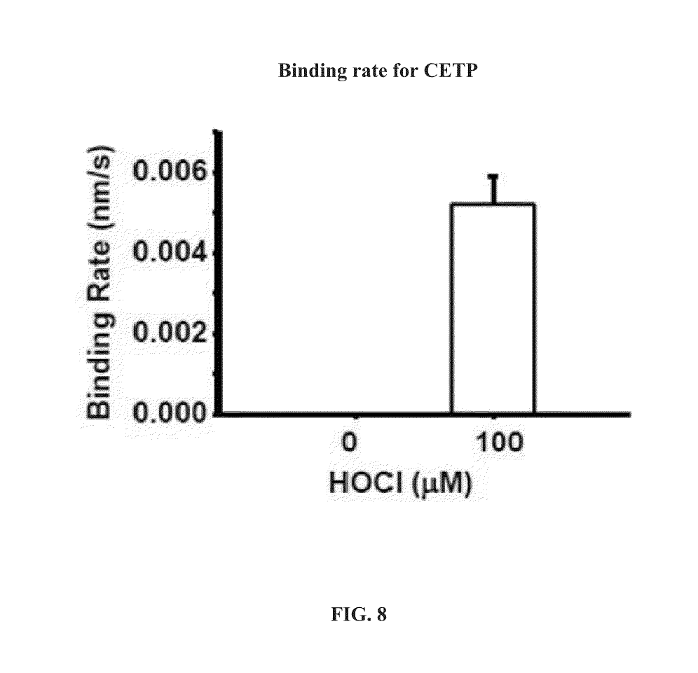

FIG. 8 shows that CETP binding rate were much slower for n-HDL than for ox-HDL. These data are consistent with reports showing that CETP binding with lipoproteins is increased by an increase in negative charges in lipoprotein lipid and protein. The effects of HOCl on CETP binding to HDL suggest that oxidation increases the negative charges.

### Example 6

Membranes from ABCA1- and SR-B1-293HEK cells appeared to fuse with HDL. To prevent this, we developed new chimeric reagents where portions of SR-B1's extracellular domain were spliced into camelid expression cassettes (Franco E J, et al., J Chromatogr B Analyt Technol Biomed Life Sci. 2010; 878:177-186; Sonneson G J, et al., Biochemistry. 2009; 48:6693-6695).

Purified human HDL (1 mg protein/mL) was labeled with biotin. Streptavidin (SA) biosensors were incubated for 15 minutes in a standard solution of the biotin-labeled HDL. The HDL-coated SA biosensors were then incubated in 100 μM HOCL in PBS or PBS alone for 15 minutes followed by a PBS wash for 3 minutes. Finally, the non-oxidized and HOCL-oxidized HDL coated SA biosensors were incubated in solutions of PON1, MPO, CETP or the chimer camelid SR-B1 (amino acids 424-434 in SRB1) for 15 minutes and rates of protein binding determined by BLI.

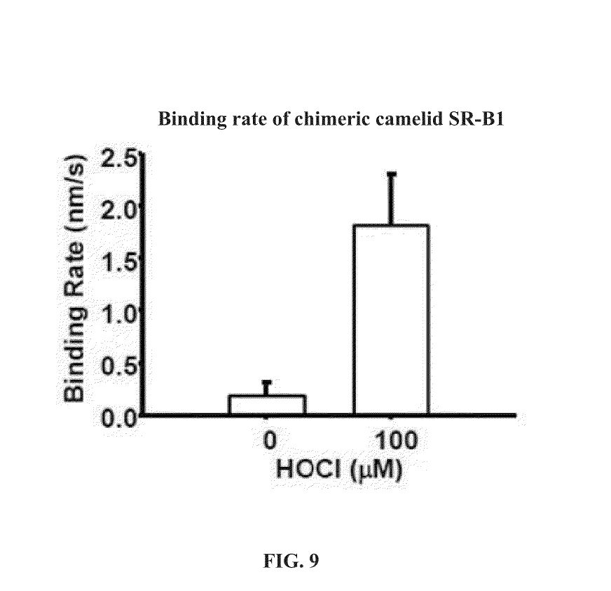

FIG. 9 shows that the n-HDL binding rate for chimeric SR-Bl-camelid protein (100 μg/mL) was much slower than for ox-HDL. These data show that BLI assays easily quantify differences in binding rates between HDL and biomolecules that are involved in inflammation or HDL metabolism. BLI assays are dynamic, not static. This makes it possible to measure the collective effects of even small changes in HDL structure, charge and molecular disorientation that influence the biophysics of HDL interactions.

BLI assays are extremely sensitive and specific for detecting dysfunctional HDL. Accordingly, BLI assays will identify human subjects that are at increased risk of atherosclerosis by accurately discriminating between plasma from controls and patients with clinically documented atherosclerosis.

### Example 7

Murine models of scleroderma and hypercholesterolemia exist (Ou J, et al., Circ Res. 2005; 97:1190-1197; Weihrauch D, et al., Am J Physiol Heart Circ Physiol. 2007; 293:141432-1441). To determine if hypercholesterolemia in Ldlr^{-/-} mice alters HDL, the binding properties of HDL in Ldlr^{-/-} mice were compared to the binding properties of HDL isolated from C57B1/6J mice using the BLI assay.

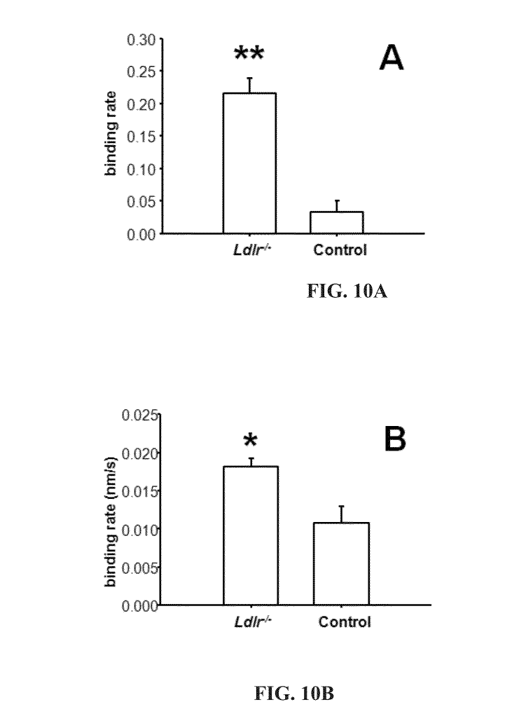

SA biosensors were incubated in a standard solution of biotin-labeled anti-mouse apolipoprotein A-I (apoA-I) antibody for 15 in plasma samples isolated from C57B1/6J and Ldlr^{-/-} mice fed western diet (6-8wk) for 15 minutes to immunocapture HDL (via apoA-I). The HDL bound anti-apoA-I biosensors were incubated in PBS as a wash to remove any non-HDL associated proteins for 3 minutes. Finally, the washed HDL-bound, anti-apoA-I-coated biosensors were incubated in standardized solutions of chimeric SR-B1 (amino acids 424-434) camelid protein in PBS (FIG. 10A) or MPO in PBS (FIG. 10B) and rates of corresponding protein binding as determined by BLI.

FIG. 10A shows that HDL binding rates for SR-B1 (aa424-434) camelid protein were increased in hypercholesterolemic Ldlr^{-/-} mice compared to binding rates for HDL in C57B1/6J mice.

FIG. 10B shows that the HDL binding rates for chimeric MPO was increased in hypercholesterolemic Ldlr^{-/-} mice compare to binding rates for HDL in C57B1/6J mice. These data provide strong support for the concept that HDL BLI assays are able to distinguish between control HDL and HDL in an established murine model of atherosclerosis.

### Example 8

Measurements of high density lipoprotein (HDL) function are reported to be more predictive for atheroprotection than measurements of HDL cholesterol. Unfortunately, bioassays of HDL function are complex, manual and time-consuming, making them impractical for routine clinical use. As such, well-standardized and easy-to-use assays of HDL function are desperately needed to make better informed clinical decisions. A panel of biolayer interferometry (BLI) assays have been established on the Octet Red (forteBio, Inc) that measure HDL binding rates for biomolecules that regulate HDL function (see Examples above). The previous examples demonstrated that hypochlorous acid-modified HDL (ox-HDL) bound paraoxonase 1, myeloperoxidase (MPO) and cholesteryl ester transfer protein at faster rates than native HDL (n-HDL). Based on these data, it was hypothesized that BLI assays of HDL binding rates might be useful for assessing functionality in vivo.

Methods: To test this hypothesis, the biophysics of HDL interactions with MPO was examined in rodent models of metabolic syndrome and hypercholesterolemia and in patients with documented cardiovascular disease (CVD). Anti-apoA-I Antibody was biotin labeled with EZ-link NHS-PEG4-Biotin from Thermo at a ratio of 1:1 (biotin:antibody).

Human HDL was purchased from Biomedical Technologies Inc.. HDL (100 μg) was oxidized with HOCl (100 μM, overnight). BLI binding study conditions HDL (40 μg/mL); MPO (100 ug/mL). Rates of binding were calculated from changes in wavelength interference with respect to time. Human data normalized to plasma HDL concentrations.

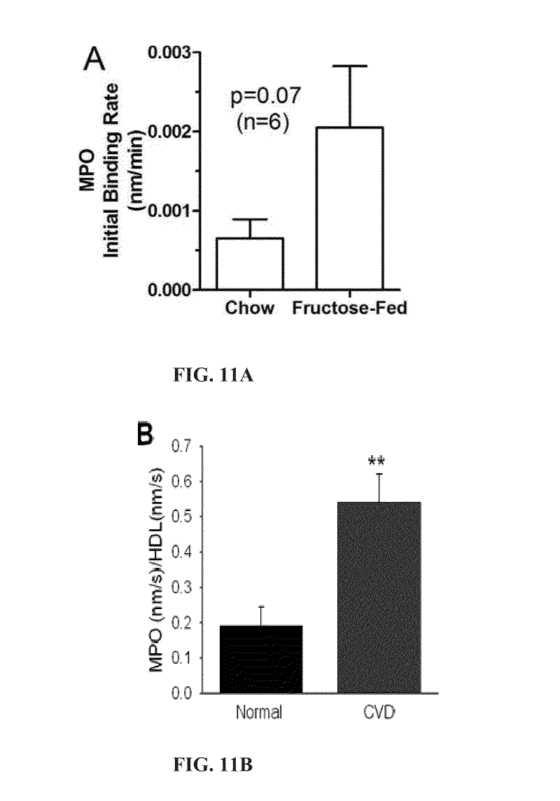

SA biosensors were incubated in a standard solution of biotin-labeled anti-mouse apolipoprotein A-I (apoA-I) antibody for 15 minutes. The anti-apoA-I-coated biosensor was washed in PBS for 3 minutes, then incubated in plasma samples isolated from chow-fed hamsters or high-fructose-fed hamsters for 15 min. The HDL bound anti-apoA-I biosensors were incubated in PBS as a wash to remove any non-HDL associated proteins for 3 minutes. The washed HDL-bound anti-apoA-I-coated biosensors were then incubated in a standard solution of MPO in PBS and rates of MPO binding determined by BLI (FIG. 11A).

SA biosensors were incubated in a standard solution of biotin-labeled anti-human apolipoprotein A-I (apoA-I) antibody for 15 minutes. The anti-apoA-I-coated biosensors were washed in PBS for 3 minutes, then incubated in plasma samples isolated from apparently healthy control subjects or patients with doctor diagnosed cardiovascular disease for 15 min. The HDL bound anti-apoA-I biosensors were incubated in PBS as a wash to remove any non-HDL associated proteins for 3 minutes. The washed HDL-bound anti-apoA-I-coated biosensors were then incubated in a standard solution of MPO in PBS and rates of MPO binding determined by BLI (FIG. 11B).

In summary, HDL was immunocaptured onto anti-apoA-I biosensors, incubated in a standardized solution of MPO and binding rates determined in ˜3.5 min/sample.

Results: HDL from fructose-fed hamsters tended to bind MPO at faster rates than from chow-fed hamsters (300% of control p<0.07, n=6) (FIG. 11A). These data provide support for the idea that metabolic syndrome induces physical changes in HDL that can be detected by a change in MPO binding rates to HDL.

HDL from patients with CVD bound MPO at faster rates than HDL from control humans (280% of control p<0.004, n=6) (FIG. 11B). These data show that HDL from CVD patients binds MPO at much higher rates than HDL from control subjects. Thus, the HDL BLI assay can be used to distinguish between apparently healthy controls and patients with atherosclerosis.

Conclusions: HDL in hamsters with metabolic syndrome and patients with CVD appears to bind MPO at faster rates than their corresponding controls. Automated BLI assays make it possible to complete large scale clinical studies in a reasonable time frame. Finally biophysical interactions of HDL with biomolecules such as MPO are a useful approach for quantifying HDL functionality.

An advantage of the methods and compositions disclosed herein is that the BLI HDL assays require no pre-labeling, can be performed in relatively short time frame (˜3 min) and are highly reproducible. Quantitative measures of HDL interactions with MPO may be a sensitive bioindicator of HDL functionality. Biophysical interactions of HDL with biomolecules involved in HDL-dependent cholesterol metabolism may be useful as sensitive bio-indicators of HDL function. The biomolecules may be directly or indirectly involved. Complexes of one or more biomolecules may be detected.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof The disclosures of patents, references and publications cited in the application are incorporated by reference herein.

What is claimed is:

1. A method comprising
(a) contacting a surface with immobilized HDL with a sample containing a biomolecule;
(b) detecting HDL-biomolecule binding on said surface; and
(c) correlating biomolecule-HDL binding with HDL function.

2. The method of claim 1, wherein the HDL is immobilized by using an antibody to HDL.

3. The method of claim 2, wherein using an antibody comprises using antibody to ApoA-1.

4. The method of claim 1, wherein said surface is the surface of a biosensor.

5. The method of claim 4, wherein said biosensor comprises a first reflecting surface comprising immobilized HDL and a second reflecting surface, which lacks immobilized HDL.

6. The method of claim 1, wherein contacting said surface with a sample comprises using a sample selected from the group consisting of blood, serum, plasma, lymph fluid, cerebrospinal fluid, peritoneal fluid, and protein preparation.

7. The method of claim 6, wherein said protein preparation comprises a protein selected from the group consisting of: PON1, PAF-AH, GPxl, MPO, XO, LCAT, CETP, SR-B1, ABCA1, ABCG1, ABCG4, CD36, Lox-1 (Lectin-like Oxidized LDL receptor) and fragments thereof. .

8. The method of claim 1, wherein said biomolecule is suspected of being an anti/pro-oxidant enzyme.

9. The method of claim 1, wherein said biomolecule is suspected of being an enzyme that metabolizes HDL cholesterol.

10. The method of claim 1, wherein detecting comprises the use of interferometry, ellipsometry, or surface plasmon resonance spectrometry.

11. The method of claim 10, wherein step (b) further comprises adding a reagent to increase the oxidation state of HDL.

12. The method of claim 1 further comprising correlating HDL-biomolecule binding with risk of atherosclerosis.

13. The method of claim 1 further comprising correlating HDL function with a risk of atherosclerosis.

14. A method for measuring binding of a biomolecule to high density lipoprotein (HDL) comprising
(a) providing a biosensor with two reflecting surfaces, wherein interference between a reflected beam from a first reflecting surface, which comprises immobilized HDL, and a reflected beam from a second reflecting surface, which lacks immobilized HDL, varies upon binding of a biomolecule to HDL;
(b) exposing said biosensor to a biomolecule;
(c) determining whether a change occurs in the interference between the reflected beams, whereby such a change is indicative of binding between the biomolecule and HDL; and
(d) determining a binding rate between the biomolecule and HDL.

15. The method of claim 14, wherein providing a biosensor comprises coating said first reflecting surface with an antibody.

16. The method of claim 15, wherein coating said first reflecting surface comprises coating with an antibody to ApoA-1.

17. The method of claim 14, wherein exposing said biosensor to a biomolecule comprises exposing said biosensor to a biological sample.

18. A method for quantifying HDL interactions comprising:
(a) providing a biosensor with a first surface comprising biotin-labeled ApoA-1 antibodies and a second surface lacking said antibodies;
(b) incubating said biosensor with a sample comprising HDL to produce immunocaptured HDL;
(c) incubating said immunocaptured HDL with a biomolecule associated with different aspects of HDL metabolism; and
(d) determining the rate of association between HDL and said biomolecule.

19. The method of claim 18 further comprising correlating the rate of association between HDL and said biomolecule with HDL function.

20. The method of claim 18 further comprising correlating the rate of association between HDL and said biomolecule with risk of atherosclerosis.
